(19) United States
(12) Patent Application Publication
(10) Pub. No.: US 2024/0177626 A1
(43) Pub. Date: May 30, 2024

(54) Device and Method for Assisting Emergency Treatment of Cervical Spine Injury

(71) Applicant: LFR International, Encino, CA (US)

(72) Inventors: Peter G. Delaney, Boca Raton, FL (US); Zachary J. Eisner, Ann Arbor, MI (US)

(21) Appl. No.: 18/521,348

(22) Filed: Nov. 28, 2023

Related U.S. Application Data
(60) Provisional application No. 63/385,417, filed on Nov. 30, 2022.

(51) Int. Cl. G09B 19/00; A61F 5/055; G16H 40/63

(52) U.S. Cl. CPC G09B 19/003; A61F 5/055; G16H 40/63

(57) ABSTRACT

A cervical collar instructional device is provided that guides lay persons through application of a cervical collar device. In some embodiments, the cervical collar instructional device includes a storage device with a plurality of sequential guidance instructions indicative of how to apply a cervical collar device. A controller may track a state parameter indicative of a current guidance instruction of the plurality of sequential guidance instructions. A controller provides context-sensitive guidance information regarding applying the cervical collar device to an output device in response to actuation of a guidance instruction input control and corresponding with the state parameter indicative of the current guidance instruction.

### RELATED APPLICATIONS

This application claims the benefit of U.S. Prov. Application Ser. No. 63/385,417 filed Nov. 30, 2022 for a “Device and Method for Assisting Emergency Treatment of Cervical Spine Injury,” which is hereby incorporated by reference in its entirety.

### BACKGROUND

Traumatic spinal cord injury (“TSCI”) is the highest contributor to years-lived-disabled resulting from road traffic injuries. Early spinal immobilization has the potential to limit neurological deficits secondary to TSCI and morbidity. In some circumstances, layperson bystanders may be available to help the injured person before emergency services personnel arrive. However, laypersons need be trained how to properly immobilize the spine. Laypersons may be trained in-person how to apply a cervical collar, but this is expensive and resource-intensive to scale-up as participants in courses. Additionally, knowledge and skills learned by participants in in-person training decays over time, reducing return on investment.

Therefore, there is a need to overcome one or more of these difficulties.

### SUMMARY

This disclosure, in at least one aspect, provides a cervical collar instructional device that is configured to provide audible point-of-care instructions. These instructions—at the point-of-care-guide the user how to correctly immobilize the spine with a cervical collar. In some embodiments, the cervical collar instructional device includes a plurality of audio tracks with step-by-step instructions on how to apply a cervical collar. The device is programmed to play audible instructions that guide the user through application of the cervical collar based on user interaction with the device. In some embodiments, the cervical collar instructional device includes a display and the device is programmed to also present a plurality of textual and/or visual instructions how to apply the cervical collar corresponding to the audible instructions. Depending on the circumstances, the cervical collar instructional device may be embedded in a cervical collar. In some cases, the audio and/or visual instructions could reference external guides, such as diagrams or flashcards showing proper placement and application of the cervical collar.

In some aspects, this disclosure provides a cervical collar instructional device that guides lay persons through application of a cervical collar device. In some embodiments, the cervical collar instructional device includes a storage device with a plurality of sequential guidance instructions indicative of how to apply a cervical collar device. The storage device may also track a state parameter indicative of a current guidance instruction of the plurality of sequential guidance instructions. A controller provides context-sensitive guidance information regarding applying the cervical collar device to an output device in response to actuation of a guidance instruction input control and corresponding with the state parameter indicative of the current guidance instruction.

According to another aspect, this disclosure provides a method of operating a cervical collar instructional device. The method may include the step of requesting help through a guidance instruction input control. A determination is made on what is the current guidance instruction of a plurality of sequential guidance instructions indicative of how to apply a cervical collar device. The current guidance instruction is conveyed through an output device.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a cervical collar kit in which a cervical collar, instructional flashcards, and a cervical collar instructional device (“CCID”) are bundled together;

FIG. 2 is a simplified block diagram of at least one embodiment of a cervical collar with an embedded cervical collar instructional device;

FIG. 3 is a front perspective view of an example cervical collar instructional device according to an embodiment;

FIG. 4 is a back perspective view of the example cervical collar instructional device shown in FIG. 3;

FIG. 5 is a front view of the example cervical collar instructional device shown in FIG. 3;

FIG. 6 is a right side view of the example cervical collar instructional device shown in FIG. 3;

FIG. 7 is a left side view of the example cervical collar instructional device shown in FIG. 3;

FIG. 8 is a top view of the example cervical collar instructional device shown in FIG. 3;

FIG. 9 is a bottom view of the example cervical collar instructional device shown in FIG. 3;

FIG. 10 is a simplified block diagram of at least one embodiment of various environments of the cervical collar instructional device; and

FIG. 11 is a simplified flow diagram of a method of using the cervical collar instructional device according to at least one embodiment.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

This disclosure addresses a number of technical problems. As discussed herein, this disclosure provides an improvement in the healthcare field with regard at least to patient safety and better patient outcomes. For example, embodiments of the cervical collar instructional device described herein guides a layperson how to correctly apply a cervical collar with audible step-by-step instructions, which can lead to earlier spinal immobilization. The early spinal immobilization that the cervical collar instructional device makes possible has the potential to limit neurological deficits secondary to traumatic spinal cord injury and morbidity.

Another technical problem addressed by one or more embodiments of the cervical collar instructional device is increased accuracy in laypersons applying the cervical collar. It is believed that laypersons applying a cervical collar using the cervical collar instructional device described herein will do so more accurately than other techniques for training laypersons how to apply a cervical collar. Applicant has conducted a study comparing a cervical collar instructional device accordingly to one or more embodiments of this disclosure with other techniques for training laypersons how to apply a cervical collar. With early spinal immobilization potentially limiting neurological deficits, the findings of the study suggest that point-of-care (“POC”) instructional modalities are more effective than in-person training at enabling laypeople to immobilize the cervical spine longitudinally.

The study was a randomized clinical trial (“RCT”) design to measure comparative effectiveness between three instructional interventions for c-spine immobilization device usage and skill retention at 1- and 2-month follow-ups. The primary outcome of interest was correct application of a cervical collar spinal immobilization device on a standardized patient, evaluated by completing all direct observation checklist requirements (1. does not move patient, 2. measures and appropriately sizes cervical collar for patient, 3. positions posterior cervical collar before positioning anterior component, 4. attaches Velcro securely) and meeting a cervical range of motion (“CROM”) device composite score threshold we previously developed for evaluating cervical movement. Secondary outcomes included time to application and reasons for incorrect cervical collar application. 265 participants consented and were randomized to one of four study arms: POC audio instructions with an embedded flashcard reference (“audio kit”) (Group 1), POC flashcard reference alone (Group 2), in-person training (Group 3), and a control group with no training or POC instructions (Group 4). Participants were randomized to either 1- or 2-month follow-ups to study skill retention for each intervention arm, with POC instructional material available at follow-up for Groups 1 and 2 given the inherent nature of POC instruction, but no re-training for Group 3. The proportion of participants correctly applying a cervical collar in the 3 intervention arms was compared with the control arm using 3 pairwise 2-sided chi-square tests of the 3 intervention arms to the control arm. In follow-ups, pairwise performance comparisons were performed of: (1) participants from 3 intervention arms at retention vs control arm at retention to identify longitudinal skill retention compared with no training and (2) participants from 2 POC intervention arms (audio kits, and instructional flashcards) at retention vs in-person training arm at retention to identify differences in skill decay at 1 and 2 months. Time to cervical collar application was compared using Wilcoxon rank sum tests for continuous variables and 2-sided Fisher exact tests for categorical demographic variables.

After 25 participants were excluded for prior instruction in management of suspected traumatic spinal cord injury (“TSCI”), 240 were included in the study, and 3 were lost to follow-up (1.25%), leaving 237 participants. Median participant age was 19 (IQR: 18,21) and by gender, 68.8% (n=163) of participants identified as women. 84.0% (n=199) had a high school diploma, 12.2% (n=29) had a bachelor's degree, 3.8% (n=9) had an advanced graduate degree. Correct cervical collar application was significantly higher during initial encounter for all three intervention arms (Groups 1-3) versus Group 4 controls (p<0.001) (Table 1).

TABLE 1
Proportion of Correct Cervical Collar Application
at Initial Encounter and 1-/2-month Follow-Up

| | Group 1 (audio | Group 2 | Group 3 (in- | Group 4 |
|---|---|---|---|---|
| | kit) | (flashcard) | person training) | (control) |
| | %, 95% CI, (n=) | %, 95% CI, (n=) | %, 95% CI, (n=) | %, 95% CI, (n=) |
| Initial encounter | 53.6% (39.9%, | 52.2% (38.9%, | 94.0% (82.5%, | 0.0% (0.0%, |
| (n = 237) | 67.9%) (n = 56) | 64.2%) (n = 66) | 98.4%) (n = 50) | 6.9%) (n = 65) |
| 1-month follow- | 64.5% (45.4, | 51.5% (33.9%, | 59.1% (36.7%, | 9.1% (2.4%, |
| up (n = 119) | 80.2) (n = 31) | 68.8%) (n = 33) | 78.5%) (n = 25) | 25.5%) (n = 30) |
| 2-month follow- | 86.9% (65.3%, | 48.6% (31.7%, | 48.1% (29.2%, | 9.4% (2.5%, |
| up (n = 118) | 96.5%) (n = 25) | 65.7%) (n = 33) | 67.6%) (n = 25) | 26.2%) (n = 35) |

Correct cervical collar application at 1- and 2-month follow-ups was also significantly higher for all three intervention arms versus Group 4 controls (p<0.001). However, at 1-month follow-up, Group 1 (audio kit) had the highest proportion of correct cervical collar applications (64.5%; 95CI: 45.4%, 80.2%, p<0.001). Similarly at 2-month follow-up, Group 1 again had the highest proportion of correct cervical collar applications (86.9%, 95CI: 65.3%, 96.5%, p<0.001). When the CROM device composite score threshold was applied to ensure restricted c-spine motion, Group 1 performed significantly better than Groups 2-4 (p<0.001). Failures were most often due to improperly measuring/sizing cervical collar (42.6%, n=101). Median time to cervical collar application was 284 seconds (IQR: 213, 351) for Group 1, 166 seconds (IQR: 137, 214) for Group 2, 49 seconds (IQR: 37, 60) for Group 3, and 89 seconds (IQR: 55, 146) for Group 4.

POC instruction enables laypeople to immobilize the cervical spine using a cervical collar. As skill retention decayed in Group 3 (in-person training) longitudinally, POC instruction using audio kits (Group 1) outperformed other instructional means while performance also increased during subsequent encounters with the POC instructional material. Our findings suggest POC instruction is superior to in-person training longitudinally for layperson bystanders, representing a scalable means of providing prehospital TSCI care.

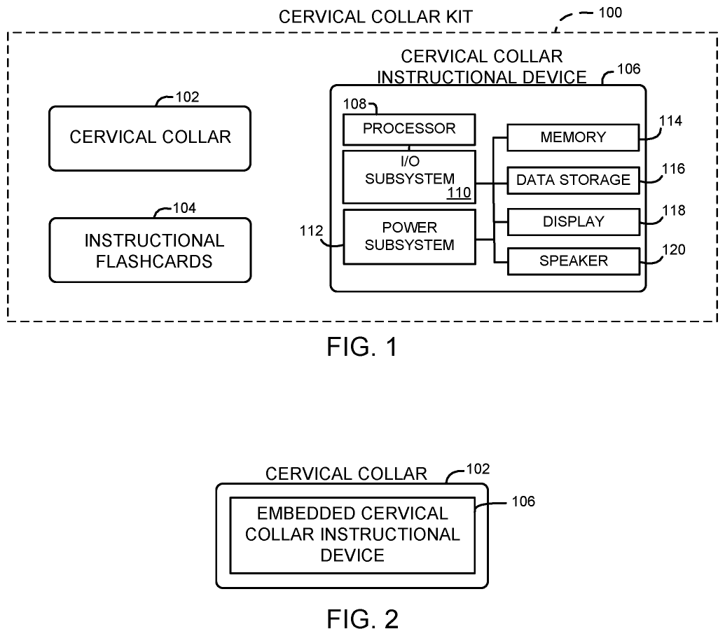

FIG. 1 illustrates an embodiment of a cervical collar kit 100 with a cervical collar 102, instructional flashcards 104, and a cervical collar instructional device (“CCID”) 106. In the embodiment shown, the cervical collar 102, instructional flashcards 104, and CCID 106 could be bundled together, such as in a box, bag, or other container. This would allow a layperson to open the kit 100, and have all the tools necessary to immobilize the injured person's spine. However, the kit 100 is shown for purposes of example only, and the CCID 106 could be distributed separately from the cervical collar 102 and/or instructional flashcards 104.

In some embodiments, one or more components of the kit 100 could be formed as part of another component. For example, the instructional flashcards 104 could be formed as part of the CCID 106. As discussed herein, the CCID 106 may include a display, which could be used to present instructional flashcards 104. In such embodiments, the instructional flashcards 104 could be optional since the visual data from the instructional flashcards 104 could be stored in the CCID 106 and presented on the CCID's 106 display. By way of another example, the CCID 106 could be embedded or otherwise incorporated into the cervical collar 102, such as diagrammatically illustrated in FIG. 2. In some such embodiments, for example, the cervical collar 102 would incorporate components of the CCID 106, such that no separate CCID 106 is needed.

Referring again to FIG. 1, the CCID 106 illustratively includes a processor 108, an input/output subsystem 110, a power subsystem 112, a memory 114, a data storage device 116, a display 118, and a speaker 120. Of course, the CCID 106 may include other components commonly found in computing devices (e.g., various input/output devices). For example, the CCID 106 could include a communication subsystem to communicate with remote devices using communication technology (e.g., wired or wireless communications) and associated protocols (e.g., Ethernet, InfiniBand®, Bluetooth®, Wi-Fi®, WiMAX, 3G, 4G LTE, etc.) to affect such communication. By way of another example, the CCID 106 could include screen mirroring and/or sharing technology to remotely display all or a portion of the information shown on the display 118 to another display, such as the user's mobile device or an external display). Likewise, audio playback on the speaker 120 could be shared with a remote device, such as the user's mobile device and/or a network-enabled speaker, for playback depending on the circumstances. Additionally, in some embodiments, one or more of the illustrative components may be incorporated in, or otherwise form a portion of, another component. For example, the memory 114, or portions thereof, may be incorporated in the processor 108 in some embodiments.

The processor 108 may be embodied as any type of processor capable of performing the functions described herein. The processor 108 may be embodied as a single or multi-core processor, a digital signal processor, microcontroller, or other processor or processing/controlling circuit. The illustrative processor 108 is configured with general-purpose processing unit(s) capable of executing programmed instructions.

The power subsystem 112 may be embodied as any type of power charging circuit or component capable of providing a power source/charging functions described herein. For example, in some embodiments, the power subsystem 112 may include one or more batteries and a charging circuit or charging components. By way of example only, in some embodiments, the power subsystem may include an interface (e.g., USB interface, etc.) for charging one or more batteries to power the electronic components of the CCID 106.

The memory 114 may be embodied as any type of volatile or non-volatile memory or data storage capable of performing the functions described herein. In operation, the memory 114 may store various data and software used during operation of the CCID 106 such as operating systems, applications, programs, libraries, and drivers. The memory 114 is communicatively coupled to the processor 108 via the I/O subsystem 110, which may be embodied as circuitry and/or components to facilitate input/output operations with the processor 108, the memory 114, and other components of the CCID 106. For example, the I/O subsystem 110 may be embodied as, or otherwise include, memory controller hubs, input/output control hubs, sensor hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 110 may form a portion of a system-on-a-chip (SoC) and be incorporated, along with the processor 108, the memory 114, and other components of the CCID 106, on a single integrated circuit chip. Similarly, the data storage device 116 may be embodied as any type of device or devices configured for short-term or long-term storage of data such as, for example, memory devices and circuits, memory cards, hard disk drives, solid-state drives, non-volatile flash memory, or other data storage devices.

The display 118 may be embodied as any type of display capable of performing the functions described herein. For example, the display 118 could be a liquid crystal display (“LCD”), a light-emitting diode (“LED”) display, an organic light-emitting diode (“OLED”) display, an active-matrix organic light-emitting diode (“AMOLED”) display, a micro LED display, or any other type of display capable of displaying the textual/graphical information to the user to guide in applying the cervical collar 102 as described herein. In some embodiments, the display 118 could be optional, and only audio instructions could be provided by the CCID 106. However, the display 118 could be helpful for cases in which hearing-impaired people utilize the device or it was being applied in a noisy environment.

The speaker 120 may be embodied as one or more speakers capable of playing sound. For example, the speaker 120 could be one or more transducers capable of playing back audio files stored on the CCID 106.

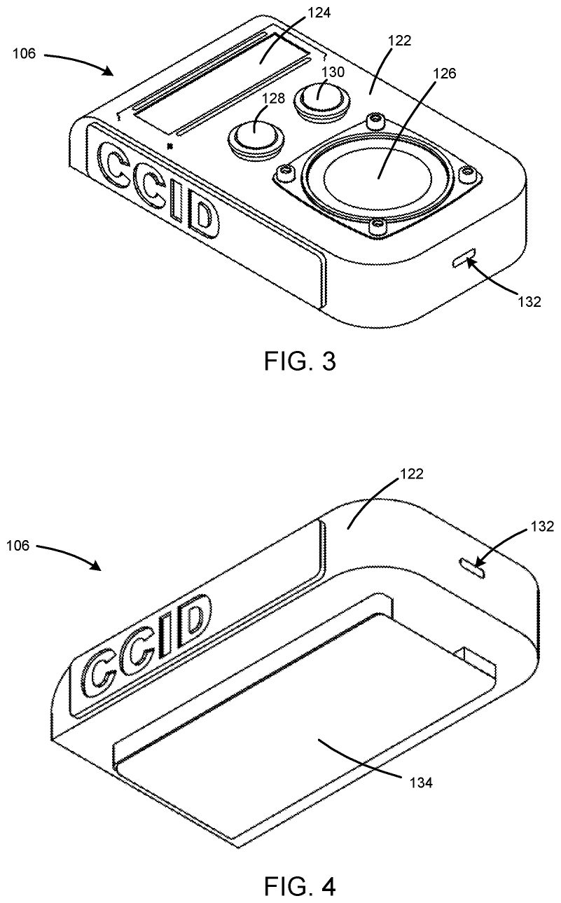

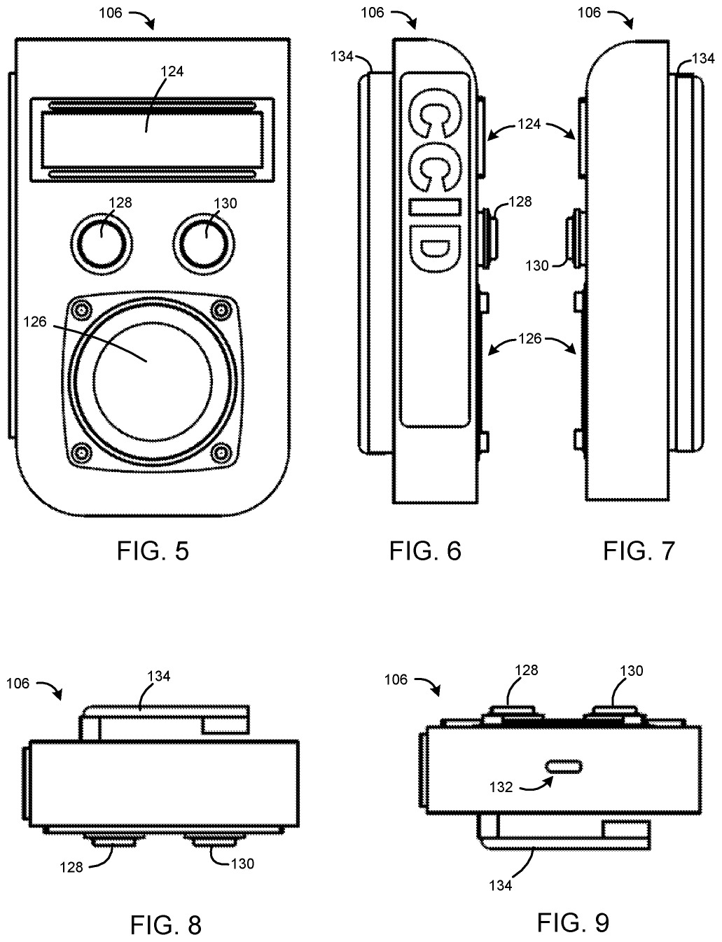

Referring now to FIGS. 3-9, there is shown an illustrative embodiment of the CCID 106. As shown, the CCID 106 includes a housing 122 for containing internal components. The housing 122 could have a wide variety of shapes and sizes depending on the circumstances. In the embodiment shown, the housing 122 includes a cutout portion to receive a display 124 for presenting textual/graphic information for the user. As shown, the housing 122 also includes a cutout to receive a speaker 126 for playing back guided audio instructions to the user on how to apply the cervical collar 102. In the embodiment shown, the housing 122 includes cutouts for a user interface, which could be embodied as a start button 128 and a continue button 130. Although two buttons are shown for purposes of example, the user interface could be hardware and/or software interface elements, such as buttons, switches, knobs, levers, soft keys, graphical user interface elements, etc., that allow the user to interact with the CCID 106. As discussed herein, the buttons 128, 130 could be actuated by the user to, among other things, advance the guided instructions, repeat instructions and/or restart the instructions. Of course, in embodiments in which the display 124 is a touchscreen, the buttons 128, 130 may be optional, and the user could instead interact with the touchscreen as the user interface. The CCID 106, in the embodiment shown, includes an interface 132 for charging the batter(ies) of the CCID 106. By way of example only, the interface 132 could be a USB-type interface for charging the CCID 106 in some cases. Depending on the circumstances, the CCID 106 may include a clip 134, such as for attaching to the cervical collar 102 and/or flashcards 104.

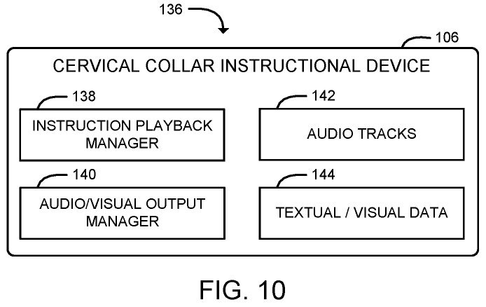

Referring now to FIG. 10, the CCID 106 establishes an environment 136 during operation to perform one or more of the actions described herein. The example environment 136 shown includes an instruction playback manager 138, an audio/visual output manager 140, audio tracks 142, and textual/visual data 144. As shown, the various components of the environment 136 may be embodied as hardware, firmware, software, or a combination thereof. As such, in some embodiments, one or more of the components of the environment 136 may be embodied as circuitry or collection of electrical devices (e.g., an instruction playback manager circuitry 138 and an audio/visual output manager circuitry 140). Additionally or alternatively, in some embodiments, those components may be embodied as hardware, firmware, or other resources of the processor 108.

In some embodiments, the instruction playback manager 138 is configured to manage which of the audio tracks 142 and/or textual/visual data 144 is presented by the CCID 106 to the user via the display 118 and/or speaker 120 based on which step in the guidance is selected. For example, the instruction playback manager 138 could be configured to sequentially present audio and/or corresponding textual/visual data to guide a user step-by-step in applying the cervical collar 102 based on the user's interaction with the buttons 128, 130 (or other user interface). For example, the audio tracks 142 could each correspond with a step in the guidance. The instruction playback manager 138 could be configured with an order to play the audio tracks 142, and step through the audio tracks 142 based on user interaction with the buttons 128, 130 (or other user interface). For example, the instruction playback manager 138 could track of which step in the guidance is the current step based on user interaction with the buttons 128, 130. In some embodiments, instead of a separate audio track for each instruction step, one or more audio tracks could be indexed so the instruction playback manager 138 is configured to move a playhead to the appropriate time/index corresponding to the instruction step to be played. Likewise, the instruction playback manager 138 could be configured to sequence through textual/visual data based on which step in the guidance is current.

The audio/visual output manager 140 is illustratively configured to output an audio track 142 selected by the instruction playback manager 138 to the speaker 120. The audio tracks 142 could be embodied as any type of digital audio file, including but not limited to MP3, AAC, WAV, etc. Likewise, the audio/visual output manager 140 is configured to output textual/visual data selected by the instruction playback manager 138 to the display 118. Depending on the circumstances, the audio/visual output manager 140 could present a multimedia instruction, such as an animation, video, etc., on the display 118. The following are examples are audio and/or textual instructions that could be presented by the audio/visual output manager 140 on the display 118 and speaker 120. Although these example instructions describe seven steps of guidance, more or less steps could be provided depending on the circumstances. The diagrams referenced in the instructions could correspond with the flashcards 104:
Device Activated. Please follow the instructions and press continue after completing each step. To hear the step again, press the start button. To start the steps from the beginning, press and hold the start button for five seconds.
Step 1: Measure patient head in neutral head position from shoulder to chin, using your hand, as depicted in step 1 diagram.
Step 2: Match your patient measurement to the corresponding collar size on the side of the collar as depicted in step 2 diagram, starting from the bottom plastic edge to the size window.
Step 3: Adjust collar and chin support to size selected in step 2 by pulling up on the chin support until the indicator is in the corresponding size window selected in step 2, as depicted in step 3 diagram.
Step 4: Lock both sides by pressing the two lock tabs, depicted in step 4 diagram, to lock the collar in place.
Step 5: With the collar now appropriately sized, pre-form the collar's chin support by bending it before applying to the patient, as depicted in step 5 diagram.
Step 6: Without moving the patient's neck, apply the collar by sliding the rear panel underneath the patient's neck, as depicted in step 6 diagram.
Step 7: Positioning the anterior chin support under the patient's chin by pulling the collar around the neck to ensure a close fit while holding the rear panel in place, as depicted in step 7 diagram. Then fasten the collar securely in place.
Application sequence completed. Please call 9-1-1 to request emergency medical services, if you or a bystander has not already done so.

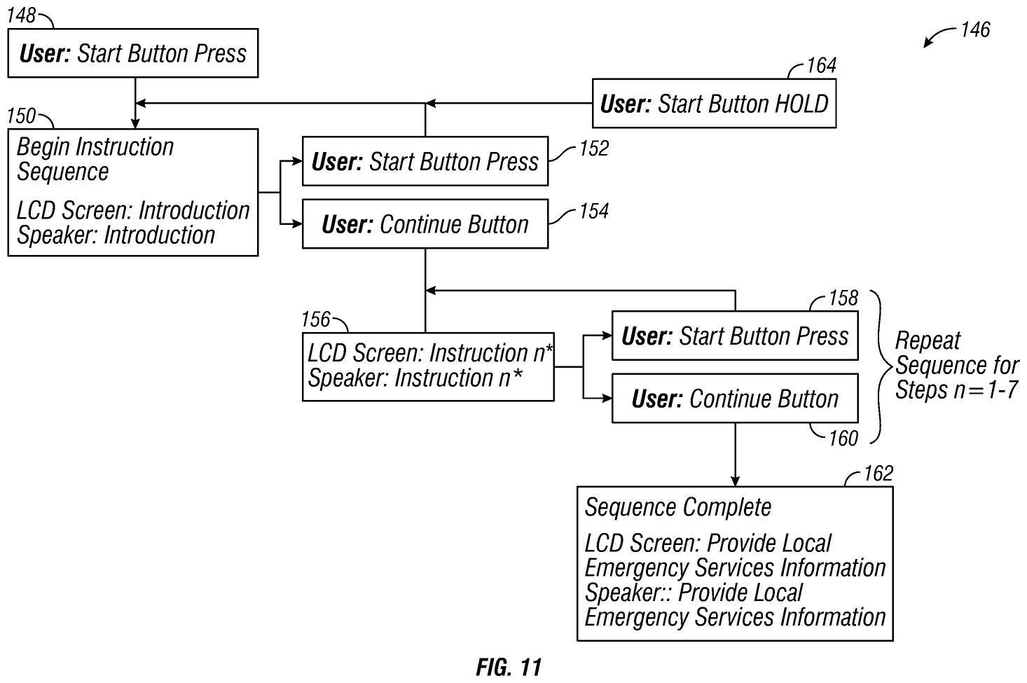

Referring now to FIG. 11, in use, the CCID 106 may execute a method 146 for guiding the user through application of the cervical collar 102. It should be appreciated that, in some embodiments, the operations of the method 146 may be performed by one or more components of the environment 136 as shown in FIG. 10, such as the instruction playback manager 138 and/or the audio/visual output manager 140. The method 146 begins in block 148 in which the user selects the start button 128. Upon activation via the start button 128, the CCID 106 advances to block 150 in which the CCID 106 will begin providing instructions for cervical collar application via the speaker 120 and/or display 118. In some embodiments, each instruction will be followed by a prompt, asking the user to press the continue button 130 when they have completed the step, or the start button 128 if they would like to hear the step once more. If the user selects the start button 128 again (block 152), the method 146 returns to block 150 and repeats the instructions via the speaker 120 and/or display 118. If the user selects the continue button 130 (block 154), the method 146 proceeds to block 156 and the next instruction in the guidance will be played/displayed via the speaker 120 and display 118. If the user selects the start button 128 again (block 158), the method 146 returns to block 156 and repeats the instructions via the speaker 120 and/or display 118. If the user selects the continue button 130 (block 160), the method 146 advances to the next instruction, and continues to operate in a similar manner until all instructions are complete, and then the method advances to block 162 in which the user will be prompted to contact emergency medical services via 9-1-1 if they wish to activate additional EMS. At any time, the user may restart the entire sequence by holding the start button for five (5) seconds (or some other predetermined period) (block 164), and the method 146 will return to block 150 to restart the instructions.

1. A cervical collar kit comprising:
a cervical collar device; and
a cervical collar instructional device comprising:
a guidance instruction input control;
an output device;
a storage device having stored thereon: (i) a plurality of sequential guidance instructions indicative of how to apply the cervical collar device; and (ii) a state parameter indicative of a current guidance instruction of the plurality of sequential guidance instructions; and
a controller to provide context-sensitive guidance information regarding applying the cervical collar device to the output device in response to actuation of the guidance instruction input control and corresponding with the state parameter indicative of the current guidance instruction.

2. The cervical collar kit of claim 1, wherein the guidance instruction input control comprises at least one button.

3. The cervical collar kit of claim 2, wherein the guidance instruction input control comprises a start button and a continue button.

4. The cervical collar kit of claim 3, wherein the controller is configured to reset the state parameter such that the current guidance instruction is set to a first guidance instruction of the plurality of sequential guidance instructions in response to actuation of the start button for at least a predetermined period of time.

5. The cervical collar kit of claim 4, wherein the controller is configured to repeat the current guidance instruction to the output device in response to actuation of the start button for less than the predetermined period of time.

6. The cervical collar kit of claim 5, wherein the controller is configured to change the state parameter to advance the current guidance instruction to a next guidance instruction of the plurality of sequential guidance instructions and provide the next guidance instruction to the output device in response to actuation of the continue button.

7. The cervical collar kit of claim 1, wherein the output device comprises at least one speaker and/or at least at least one display.

8. The cervical collar kit of claim 1, wherein the cervical collar instructional device includes a housing with a clip dimensioned to receive the cervical collar device.

9. A cervical collar instructional device to provide guidance on applying a cervical collar device, the cervical collar instructional device comprising:
a guidance instruction input control;
an output device;
a storage device having stored thereon: (i) a plurality of sequential guidance instructions indicative of how to apply a cervical collar device; and (ii) a state parameter indicative of a current guidance instruction of the plurality of sequential guidance instructions; and
a controller to provide context-sensitive guidance information regarding applying the cervical collar device to the output device in response to actuation of the guidance instruction input control and corresponding with the state parameter indicative of the current guidance instruction.

10. The cervical collar instructional device of claim 9, wherein the guidance instruction input control comprises at least one button.

11. The cervical collar instructional device of claim 10, wherein the guidance instruction input control comprises a start button and a continue button.

12. The cervical collar instructional device of claim 11, wherein the controller is configured to reset the state parameter such that the current guidance instruction is a first guidance instruction of the plurality of sequential guidance instructions in response to actuation of the start button for at least a predetermined period of time.

13. The cervical collar instructional device of claim 12, wherein the controller is configured to repeat the current guidance instruction to the output device in response to actuation of the start button for less than the predetermined period of time.

14. The cervical collar instructional device of claim 13, wherein the controller is configured to change the state parameter to advance the current guidance instruction to a next guidance instruction of the plurality of sequential guidance instructions and provide the next guidance instruction to the output device in response to actuation of the continue button.

15. The cervical collar instructional device of claim 9, wherein the output device comprises at least one speaker and/or at least at least one display.

16. The cervical collar instructional device of claim 9, wherein the cervical collar instructional device includes a housing with a clip dimensioned to receive a cervical collar device.

17. A method of operating a cervical collar instructional device, the method comprising:
requesting help through a guidance instruction input control;
determining a current guidance instruction of a plurality of sequential guidance instructions indicative of how to apply a cervical collar device; and
conveying through an output device one or more instructions on applying a cervical collar device corresponding to the current guidance instruction.

18. The method of claim 17, wherein in response to detecting a subsequent actuation of the guidance instruction input control, either (i) re-convey through the output device one or more instructions corresponding to the current guidance instruction; or (ii) advance to a next guidance instruction of the plurality of sequential guidance instructions.

19. The method of claim 18, wherein in response to detecting an actuation of the guidance instruction input control for more than a predetermined time period, resetting the current guidance instruction to a first guidance instruction of the plurality of sequential guidance instructions.

20. The method of claim 19, wherein the output device comprises at least one speaker and/or at least at least one display.
